(19) United States
(12) Patent Application Publication
(10) Pub. No.: US 2023/0322812 A1
(43) Pub. Date: Oct. 12, 2023

(54) CRYSTALLINE FORM OF HETEROBICYCLIC COMPOUND

(71) Applicants: TAIHO PHARMACEUTICAL CO., LTD., Tokyo (JP); OTSUKA PHARMACEUTICAL CO., LTD., Tokyo (JP)

(72) Inventors: Takashi MITA, Tokyo (JP); Daisuke TAKEDA, Tokyo (JP)

(73) Assignees: TAIHO PHARMACEUTICAL CO., LTD., Tokyo (JP); OTSUKA PHARMACEUTICAL CO., LTD., Tokyo (JP)

(21) Appl. No.: 18/042,503

(22) PCT Filed: Aug. 24, 2021
(86) PCT No.: PCT/IB2021/057741

§ 371 Date: Feb. 22, 2023

(30) Foreign Application Priority Data
Aug. 24, 2020 (JP) 2020-140877

(51) Int. Cl. C07D 519/00

(52) U.S. Cl. CPC C07D 519/00; C07B 2200/13

(57) ABSTRACT

An object of the present invention is to provide a crystal of compound 1, which is useful as an antitumor agent, the crystal being stable, excellent in oral absorbability, highly chemically pure, and suitable for mass production. The present invention provides a crystal of compound 1 that exhibits an X-ray powder diffraction spectrum containing, at diffraction angles 2θ±0.2°, characteristic peaks of at least 3 characteristic peaks selected from the group consisting of 5.8°, 9.8°, 12.2°, 14.0°, 15.3°, 21.1°, and 27.6°.

### TECHNICAL FIELD

The present invention relates to novel crystals of a heterobicyclic compound that are stable, excellent in oral absorbability, and useful as an antitumor agent.

### BACKGROUND ART

Pharmaceutical compositions for oral administration are typically required to exhibit not only stability of the active ingredient, but also excellent absorbability during oral administration; mass production methods of the compositions are also required.

In crystals, there may be polymorphs that contain the same molecule but have different molecular arrangements. Such polymorphs are known to exhibit different peaks in X-ray powder diffraction measurement (XRPD measurement). Additionally, those crystal polymorphs are known to exhibit different solubility, oral absorbability, stability, and the like. Thus, optimal crystals must be found from different perspectives in developing drugs.

At present, a number of SHP2 inhibitors are reported as antitumor agents, and Patent

Literature 1 discloses 241R,2R,4S)-2-amino-7-azabicyclo[2.2.1]heptan-7-yl)-5-(3,4dichloro-2-methyl-2H-in dazol-5-yl)-3-methyl-3,7-dihydro-4Hpyrrolo[2,3-d]pyrimidin-4-one (free base, hereinafter “compound 1”), which is a compound that has excellent SHP2 inhibitory activity and that exhibits antitumor activity.

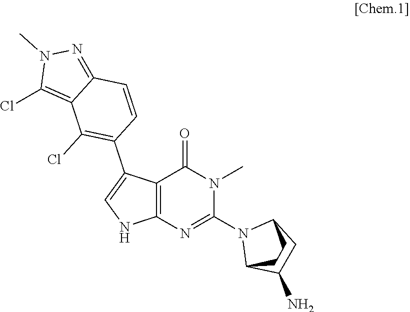 [SMILES: Cn1nc2ccc(-c3c[nH]c4nc(N5[C@H]6CC[C@@H]5[C@H](N)C6)n(C)c(=O)c34)c(Cl)c2c1Cl]

compound 1

However, Patent Literature 1 does not disclose or suggest the crystal of compound 1, as well as the stability, oral absorbability, and crystallization method of compound 1.

### CITATION LIST

### Patent Literature

Patent Literature 1: WO2020/022323 pamphlet

### SUMMARY OF INVENTION

### Technical Problem

An object of the present invention is to provide a crystal of compound 1, which is useful as an antitumor agent, the crystal being stable, excellent in oral absorbability, and suitable for mass production; and to provide a method for crystallizing compound 1.

### Solution to Problem

The present inventors conducted extensive research, and found that the free base of compound 1 has a crystalline form.

Specifically, the present invention provides the following Items.

Item 1

A crystal of 2-((1R,2R,4S)-2-amino-7-azabicyclo[2.2.1]heptan-7-yl)-5-(3,4-dichloro-2-methyl-2Hi ndazol-5-yl)-3-methyl-3,7-dihydro-4H-pyrrolo[2,3-d]pyrimidin-4-one exhibiting an X-ray powder diffraction spectrum containing, at diffraction angles 20±0.2°, at least 3 characteristic peaks selected from the group consisting of 5.8°, 9.8°, 12.2°, 14.0°, 15.3°, 21.1°, and 27.6°.

Item 2

The crystal according to item 1, exhibiting an X-ray powder diffraction spectrum containing, at diffraction angles 20±0.2°, characteristic peaks of 5.8°, 9.8°, 12.2°, 14.0°, 15.3°, 21.1°, and 27.6°.

Item 3

The crystal according to item 1 or 2, which has a chemical purity of 98.0% or more.

Item 4

The crystal according to any one of items 1 to 3, which has an x-ray powder diffraction spectrum substantially as shown in [FIG. 1].

Item 5

The crystal according to any one of items 1 to 4, which has an endothermic peak of 305±10° C. as measured by differential scanning calorimetry (DSC).

Item 6

The crystal according to any one of items 1 to 5, which has a DSC curve substantially as shown in [FIG. 2].

Item 7

The crystal according to any one of items 1 to 6, which has a dynamic vapor sorption (DVS) curve substantially as shown in [FIG. 3].

Item 8

A pharmaceutical combination comprising the crystal according to any one of items 1 to 7 and one or more other therapeutic agents.

Item 9

A pharmaceutical combination according to item 8, wherein the one or more other therapeutic agents are selected from anti-cancer agents or adjuvants (supporting agents in the therapy) in cancer therapy.

Item 10

A pharmaceutical composition comprising the crystal according to any one of items 1 to 7 or a pharmaceutical combination according to item 8 or 9 and a pharmaceutically acceptable excipient.

Item 11

The crystal according to any one of items 1 to 7, pharmaceutical combination according to item 8 or 9 or pharmaceutical composition according to item 10 or for use in medicine.

Item 12

The crystal, pharmaceutical combination or pharmaceutical composition for use according to item 11, for use in the treatment of cancer.

Item 13

The crystal, pharmaceutical combination or pharmaceutical composition for use according to item 12, wherein the cancer is head and neck cancer, esophagus cancer, gastric cancer, colon cancer, rectum cancer, liver cancer, gallbladder cancer, cholan-giocarcinoma, biliary tract cancer, pancreatic cancer, lung cancer, breast cancer, ovarian cancer, cervical cancer, endometrial cancer, renal cancer, bladder cancer, prostate cancer, testicular tumor, osteosarcoma, soft-tissue sarcoma, leukemia, myelodysplastic syndrome, chronic myeloproliferative disease, malignant lymphoma, multiple myeloma, skin cancer, brain tumor, or mesothelioma.

Item 14

A method for producing the crystal of any one of items 1 to 7, comprising a crystallization step using at least one solvent selected from the group consisting of acetonitrile, methanol, ethanol, 1-propanol, 2-propanol, ethyl acetate, toluene, tetrahydrofuran, tert-butyl methyl ether, isopropyl ether, DMSO and water.

Item 15

The method according to item 14, wherein the crystallization step is performed using dimethyl sulfoxide as a good solvent and water as a poor solvent.

### Advantageous Effects of Invention

The crystal of compound 1 (hereinafter, “crystal I”) of the present invention exhibits high stability, excellent oral absorbability, high crystallinity, and high chemical purity, and is also suitable for mass production, while having homogenous particle size dis-tribution. Crystal I can also be obtained by adding a specific solvent to compound 1 to crystallize it. Thus, crystal I can be used as an orally administered drug.

### BRIEF DESCRIPTION OF DRAWINGS

FIG. 1 shows a powder X-ray powder diffraction spectrum of crystal I of compound 1 in free base form. The vertical axis represents intensity (cps). The horizontal axis represents a diffraction angle 20)(°.

FIG. 2 shows a differential scanning calorimetry (DSC) curve of crystal I of compound 1. The vertical axis represents DSC (W/g). The horizontal axis represents a temperature (° C.).

FIG. 3 shows a dynamic vapor sorption (DVS) curve of crystal I of compound 1. The vertical axis represents a weight change rate (%). The horizontal axis represents a relative humidity (% RH).

### DESCRIPTION OF EMBODIMENTS

Compound 1 of the present invention in an amorphous solid form can be synthesized by the production method disclosed in Patent Literature 1.

A crystal refers to a solid, in which atoms or molecules are arranged in an orderly repeating pattern, and differs from an amorphous solid, which does not have repeating units. Crystals and amorphous solids can be examined by methods such as X-ray powder diffraction measurement (XRPD measurement), differential scanning calorimetry measurement (DSC measurement), thermogravimetric measurement-differential thermoanalysis (TG-DTA), dynamic vapor sorption (DVS), or IR spec-troscopy (IR).

In crystals, there may be polymorphs that contain the same molecule but have different molecular arrangements. Such polymorphs are known to exhibit peaks different from other polymorphs in X-ray powder diffraction measurement (XRPD measurement). Such polymorphs are also known to exhibit different solubility, oral absorbability, stability, and the like. Thus, optimal crystals must be found from different perspectives in developing drugs.

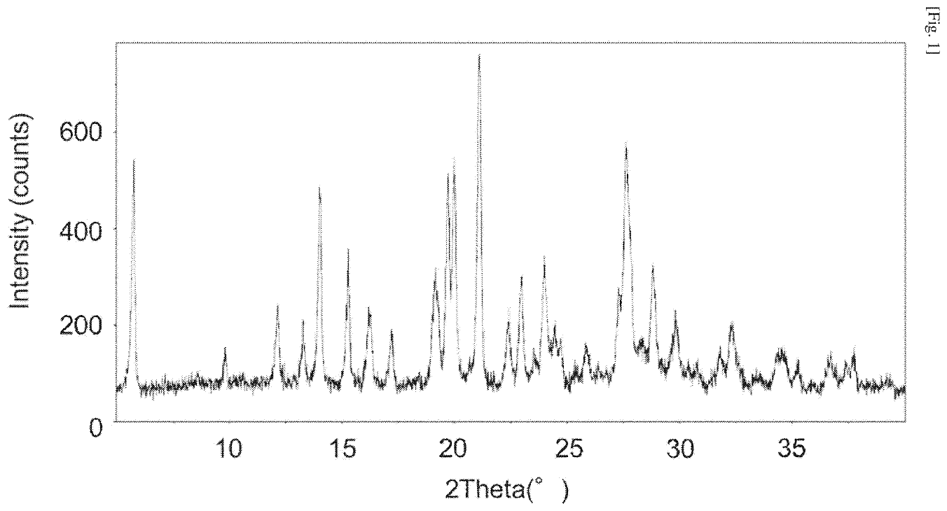

The X-ray powder diffraction spectrum of crystal I of the present invention contains at least 3 characteristic peaks selected from the group consisting of 5.8°, 9.8°, 12.2°, 14.0°, 15.3°, 21.1°, and 27.6° at diffraction angles 20±0.2°, preferably contains at least 5 characteristic peaks selected from the group consisting of 5.8°, 9.8°, 12.2°, 14.0°, 15.3°, 21.1°, and 27.6° at diffraction angles 20±0.2°, preferably contains the characteristic peaks of 5.8°, 9.8°, 12.2°, 14.0°, 15.3°, 21.1°, and 27.6° at diffraction angles 20±0.2°, and more preferably contains the characteristic peaks of 5.8°, 9.8°, 12.2°, 13.3°, 14.0°, 15.3°, 16.3°, 17.2°, 19.1°, 19.7°, 20.0°, 21.1°, 23.0°, 24.0°, 24.5°, 27.3°, 27.6°, 28.8°, 29.8°, and 32.2° at diffraction angles 20±0.2°. Particularly preferably, the X-ray powder diffraction spectrum of crystal I of the present invention is as shown in [FIG. 1].

In one embodiment, the X-ray powder diffraction spectrum of crystal I of the present invention contains characteristic peaks at 14.0°, 21.1°, and 27.6° at diffraction angles 2θ±0.2°.

In one embodiment, the X-ray powder diffraction spectrum of crystal I of the present invention at least 3 characteristic peaks selected from the group consisting of 5.8°, 9.8°, 12.2°, 13.2°, 14.0°, 15.3°, and 16.3° at diffraction angles 2θ±0.2°, preferably contains at least 5 characteristic peaks selected from the group consisting of 5.8°, 9.8°, 12.2°, 13.2°, 14.0°, 15.3°, and 16.3° at diffraction angles 2θ±0.2°, and more preferably contains the characteristic peaks of 5.8°, 9.8°, 12.2°, 13.2°, 14.0°, 15.3°, and 16.3° at diffraction angles 2θ±0.2°.

Crystal I of the present invention includes those with different crystal habits (i.e., different external shapes) due to different growth of the crystal surface. Thus, crystal I includes crystals that exhibit different relative intensities of peaks, even if the peak patterns at diffraction angle 2θ of crystal I determined by XRD measurement are the same. The relative intensity used here refers to a value of each peak area relative to the largest peak area (taken as 100), among peaks at diffraction angle 2θ, in an X-ray powder diffraction spectrum.

The error of peaks at diffraction angle 2θ in an X-ray powder diffraction spectrum in the present invention is about ±0.2°. This is an error caused by the devices used in measurement, sample adjustment, methods of data analysis, etc. Thus, the XRD measurement values of the crystals of the present invention include an error ±0.2° in the values at diffraction angle 2θ.

DSC measurement of crystal I detects an endothermic peak at about 305° C.±10° C. (highest peak value). There are no endothermic or exothermic peak which indicates the change of crystalline form below 280° C. This indicates that crystal I is stable under heating and stirring conditions during the crystallization process or the process of preparing a suitable dosage form. Because of the excellent oral absorbability and stability, crystal I is suitable as a crystal for drugs.

The endothermic peak (the highest peak value) measured by DSC may vary depending on the temperature increase rate per minute, the weight of the sample, the purity of the sample, and other factors. Thus, the highest peak value of the crystals of the present invention as measured by DSC includes an error ±5.0° C. In this specification, the term “in the vicinity of” means±5.0° C.

Crystal I may be a monocrystal of crystal I, or a polymorphic mixture that contains other crystals. Specifically, crystal I in a polymorphic mixture form contains preferably 90 wt % or more of crystal I, more preferably 95 wt % or more of crystal I, and particularly preferably 99 wt % or more of crystal I.

In this specification, the term “chemical purity” refers to a purity measured by high-performance liquid chromatography, and a chemical purity of compound 1 indicates a purity determined by measuring compound I by high-performance liquid chromatography. The wavelength of the detector for use in purity measurement can be suitably determined. Specifically, the chemical purity of a crystal of compound 1 is preferably 95.0% or more, more preferably 98.0% or more, and particularly preferably 99.0% or more.

Crystal I of the present invention can be obtained by adding compound 1 to a specific solvent, and stirring the mixture to crystallize compound 1. Thus, the present invention provides a crystallization method to provide crystal I, the method comprising: step (1) of adding compound 1 to a solvent; and step (2) of stirring the solvent to which compound 1 has been added in step (1) to crystallize compound 1.

Compound I can be prepared according to Example 93 of WO2020/022323. Compound I can be purified by reverse phase HPLC, followed by extraction with CHCl₃—MeOH to be subjected to steps (1) and (2) above.

Solvents usable for crystallization to obtain crystal I of the present invention include lower alcohols, such as methanol, ethanol, 1-propanol, 2-propanol, and n-butanol, esters such as methyl acetate, ethyl acetate, n-propyl acetate, isopropyl acetate, n-butyl acetate, isobutyl acetate, and tert-butyl acetate, ether such as diethyl ether, diisopropyl ether, methyl tert-butyl ether, dioxane, and tetrahydrofuran (THF), acetonitrile, dimethylsulfoxide (DMSO), water, etc. In one embodiment, the solvent is 1-Propanol. The solvent is used singly or in a combination of two or more. In one embodiment, a combination of two solvents is used, wherein one solvent acts as the solvent (i.e. the good solvent) and one solvent as the anti-solvent (i.e. the poor solvent). The solvent is more preferably alcohols, DMSO and water. In one embodiment when a combination of two solvents is used, the solvent is DMSO and the anti-solvent is water. The amount of solvent (w/v) is preferably not less than 5 times and not more than 50 times the amount of compound 1, more preferably not less than 10 times and not more than 50 times the amount of compound 1.

In one aspect, the invention provides crystal I obtainable by a method comprising the steps of:
step (1) adding compound 1 to a solvent e.g. 1-propanol; and step (2) of stirring the solvent to which compound 1 has been added in step (1) to crystallize compound 1.

The temperature for crystallization to obtain crystal I of the present invention can suitably be determined according to the solvent for use within the range of 0° C. to the boiling point of the solvent. The temperature for crystallization does not necessarily stay at the same temperature, and heating or cooling can be performed between 0° C. to the boiling point of the solvent. Heating used here means maintaining the temperature of the solvent at 40° C. or more, and cooling means maintaining the temperature of the solvent at less than 15° C.

Stirring for crystallization to obtain crystal I of the present invention can suitably be performed using a stirrer, a stirring blade, a magnetic stirrer, or other stirrers, according to the amount of the solvent, and the size of the reaction furnace, and the like. The stirring rate is typically 1 to 600 rpm, and preferably 10 to 300 rpm.

The stirring time for crystallization to obtain crystal I of the present invention is preferably a predetermined length of time or more in order to sufficiently facilitate the crystallization and to obtain the crystal at high yield, and preferably less than a predetermined length of time in order to reduce the decomposition of the crystal, which decreases the yield. The stirring time is 1 minute to 120 hours, preferably 1 hour to 72 hours, and more preferably 3 hours to 48 hours.

Crystal I of the present invention precipitated in a solvent can be isolated and purified by known separation and purification techniques, such as filtration, washing with an organic solvent, and drying under reduced pressure. The organic solvents for use in washing include the solvents described above; the organic solvents are preferably ethanol, isopropanol, acetone, ethyl acetate, a mixture solvent of water-DMSO, and a mixture solvent of water-ethanol. The atmospheric pressure for drying under reduced pressure is 0.1 atm or less, and preferably 0.05 atm or less. The temperature for drying under reduced pressure is 0 to 200° C., and preferably 25 to 100° C.

For crystallization in the present invention, crystal I may be added as a seed crystal. The seed crystal to be added is 0.1 to 10 wt %, and preferably 1 to 3 wt % of the theoretical yield of crystallized compound 1.

The thus-obtained crystal I of compound 1 exhibits an X-ray powder diffraction spectrum containing, at diffraction angles 20±0.2°, at least 3 characteristic peaks selected from the group consisting of 5.8°, 9.8°, 12.2°, 14.0°, 15.3°, 21.1°, and 27.6°. Preferably, the crystal I of compound 1 exhibits an X-ray powder diffraction spectrum containing, at diffraction angles 20±0.2°, at least 5 characteristic peaks selected from the group consisting of 5.8°, 9.8°, 12.2°, 14.0°, 15.3°, 21.1°, and 27.6°. More preferably, the crystal I of compound 1 exhibits an X-ray powder diffraction spectrum containing, at diffraction angles 20±0.2°, characteristic peaks of 5.8°, 9.8°, 12.2°, 14.0°, 15.3°, 21.1°, and 27.6°.

Because of the excellent SHP2 inhibitory activity of compound 1, crystal I of the present invention is useful for use in medicine, and in particular as an antitumor agent. Although not particularly limited to, examples of the target cancer include head and neck cancer, esophagus cancer, gastric cancer, colon cancer, rectum cancer, liver cancer, gallbladder cancer, cholangiocarcinoma, biliary tract cancer, pancreatic cancer, lung cancer, breast cancer, ovarian cancer, cervical cancer, endometrial cancer, renal cancer, bladder cancer, prostate cancer, testicular tumor, osteosarcoma, soft-tissue sarcoma, leukemia, myelodysplastic syndrome, chronic myeloproliferative disease, malignant lymphoma, multiple myeloma, skin cancer, brain tumor, mesothelioma, and the like.

To use crystal I of the present invention as a drug, a pharmaceutical carrier (i.e. a pharmaceutically acceptable excipient) may optionally be added to crystal I to prepare a suitable dosage form (i.e. a pharmaceutical composition) according to the prevention or treatment purpose. Examples of the dosage form include oral drugs, injectable agents, suppositories, ointments, and patches, with oral drugs being preferable. These dosage forms can be prepared by methods known to a person skilled in the art.

The pharmaceutical carrier for use includes a range of organic or inorganic carrier substances commonly used for drug materials; and the carrier is added as an excipient, binder, disintegrant, lubricant, or colorant to solid drugs, or as a solvent, solubilizing agent, suspending agent, isotonic agent, buffer, or soothing agent to liquid drugs. Pharmaceutical preparation additives, such as preservatives, antioxidants, colorants, sweeteners, and stabilizers, may also optionally be added.

Oral solid drugs can be prepared by adding an excipient, optionally together with an excipient, binder, disintegrant, lubricant, colorant, taste-masking or flavoring agent, etc., to crystal I of the present invention to produce tablets, coated tablets, granules, powders, capsules, or the like in accordance with an ordinary method.

Injectable agents can be prepared by adding a pH adjuster, buffer, stabilizer, isotonic agent, local anesthetic, etc., to crystal I of the present invention, and processing the mixture to a subcutaneous, intramuscular, or intravenous injectable agent in accordance with an ordinary method.

The amount of crystal I of the present invention to be added to each unit of dosage form varies depending on the symptoms of the patient who is administered the drug or the dosage form. However, the desirable amount per unit of dosage form is typically 0.05 to 1000 mg for oral drugs, 0.01 to 500 mg for injectable agents, and 1 to 1000 mg for suppositories.

The daily dosage of a drug in dosage forms described above varies depending on the symptoms, body weight, age, gender of the patient, and so on; and cannot be gen-eralized. However, the dosage based on the content of crystal I of the present invention is typically 0.05 to 5000 mg, and preferably 0.1 to 1000 mg, per adult (body weight: 50 kg) per day, and it is preferable to administer the drug in one dose or in about two to three divided doses per day.

Crystal I may be administered alone i.e. as a monotherapy or as the sole active ingredient, and in particular the sole active ingredient for treating a cancer. Alternatively, the invention also provides a pharmaceutical combination comprising crystal I and one or more other therapeutic agents i.e. crystal I may be used as part of a combination therapy.

A combination therapy comprises the use of a combination of two or more compounds/agents including crystal I. In other words, in a combination therapy the two or more compounds/agents (the pharmaceutical combination) are administered as part of the same overall treatment regimen. As such, the posology of each of the two or more compounds/agents may differ: each may be administered at the same time or at different times. It will therefore be appreciated that the compounds/agents of the pharmaceutical combination may be administered sequentially (e.g. before or after) or simultaneously, either in the same pharmaceutical formulation (i.e. together), or in different pharmaceutical formulations (i.e. separately). Simultaneously in the same formulation is as a unitary formulation whereas simultaneously in different pharmaceutical formulations is non-unitary. The posologies of each of the two or more compounds/agents in a combination therapy may also differ with respect to the route of administration.

In one embodiment of the pharmaceutical combination, the one or more other therapeutic agents are selected from anti-cancer agents or adjuvants (supporting agents in the therapy) in cancer therapy.

In particular, the anti-cancer agents is selected from the group consisting of:
i. a tyrosine kinase inhibitor,
ii. a RAS-MAPK pathway inhibitor,
iii. a PI3K pathway inhibitor,
iv. a BCL2 inhibitor,
v. a CDK4/6 inhibitor,
vi. an HDAC inhibitor,
vii. a topoisomerase inhibitor (a topoisomerase I inhibitor, and topoisomerase II inhibitor),
viii. an alkylating agent,
ix. an anthracycline antibiotic,
x. an alkaloid,
xi. an anti-metabolite,
xii. an anti-microtubule agent,
xiii. a platinum-containing drug,
xiv. a proteasome inhibitor, and
xv. a thalidomide analog drug.

In one embodiment, the anti-cancer agent is selected from AKT inhibitor, ALK inhibitor, Bc12 inhibitor, BCR-ABL inhibitor, BRAF inhibitor, CDK4/6 inhibitor, CDK inhibitor, EGFR inhibitor, Erk1/2 inhibitor, FGFR inhibitor, FLT3 inhibitor, HER2 inhibitor, MEK inhibitor, Multi-kinase inhibitor, PI3K inhibitor, RAF inhibitor, HDAC inhibitor, topoisomerase I inhibitor, topoisomerase II inhibitor, alkylating agent, anthracycline antibiotics, alkaloid, anti-metabolite, anti-microtubule agent, platinum-containing drug, and proteasome inhibitor.

In one embodiment, the anti-cancer agent is selected from the group consisting of cetuximab, MK-2206, alectinib, crizotinib, venetoclax, imatinib, dasatinib, ponatinib, dabrafenib, vemurafenib, sorafenib, palbociclib, abemaciclib, osimertinib, gefitinib, erlotinib, afatinib, brigatinib, ulixertinib, (2R)-2-(6-{5-chloro-2-Roxan-4-yl)aminolpyrimidin-4-yl1-1-oxo-2,3-dihydro-1H-isoin do1-2-yl)-N-[(1S)-1-(3-fluoro-5-methoxyphenyl)-2-hydroxyethyl]propanamide or a salt thereof, erdafitinib, giltertinib, lapatinib, neratinib, trametinib, cobimetinib, binimetinib, regorafenib, sunitinib, nintedanib, anlotinib, vandetanib, lenvatinib, alpelisib and idelalisib/CAL-101, vorinostat(SAHA), irinotecan(SN-38), etoposide, cy-clophosphamide, doxorubicin, gemcitabine, pemetrexed, 5-FU, FdUrd, FTD, pa-clitaxel, cisplatin, oxaliplatin, bortezomib, afuresertib, trans-3-amino-1-methyl-3-(4-(3-phenyl-5H-imidazo[1,2-c]pyrido[3,4-e][1,3]oxazin-2-yl)phe nyl)cyclobutanol, capivasertib, ipatasertib, triciribine, miransertib, lorlatinib, ceritinib, repotrectinib, ensartinib, alkotinib, WX-0593, SAF-189s, CT-707, TQ-B3101, sabutoclax, apogossypol, obatoclax, navitoclax, APG-2575, APG-1252, asciminib, olverembatinib, encorafenib, lifirafenib, LXH-254, ribociclib, lerociclib, trilaciclib, alvocidib, GLR-2007, SHR-6390, XZP-3287, BPI1178, PF-06873600, NUV-422, FCN-437, seliciclib, mevociclib, milciclib, fadraciclib, zotiraciclib, dinaciclib, samu-raciclib, voruciclib, FIT-039, PF-07104091, BEY-1107, panitumumab, sutetinib, allitinib, epitinib, xiliertinib, rociletinib, dacomitinib, simotinib, olmutinib, yinlitinib, mefatinib, alflutinib, almonertinib, icotinib, naquotinib, poziotinib, epertinib, sapitinib, cipatinib, tarloxotinib, pyrotinib, pirotinib, lazertinib, varlitinib, tesevatinib, canertinib, mobocertinib, duligotuzumab, olafertinib, zorifertinib, pelitinib, DZD-9008, ASK120067, BPI-7711, QLNC-120, ametumumab, imgatuzumab, amivantamab, seribantumab, nimotuzumab, serclutamab, depatuxizumab, tomuzotuximab, SCT-200, ravoxertinib, LY3214996, MK-8353, LTT462, HH-2710, infigratinib, pemigatinib, orantinib, derazantinib, roblitinib, rogaratinib, zoligratinib, E-7090, AZD-4547, ODM-203, ICP-192, HMPL-453, bemarituzumab, quizartinib, crenolanib, flysyn, mi-vavotinib, PHI-101, MEN-1703, FF-10101, HM-43239, E-6201, ENMD-2076, larotinib, tucatinib(irbinitinib), BDTX-189, trastuzumab, pertuzumab, zanidatamab, zenocutuzumab, margetuximab, KN-026, BAT-8001, TAA-013, KL-A166, selumetinib, refametinib, mirdametinib, pimasertib, HL-085, NFX-179, nilotinib, dovitinib, axitinib, vatalinib, pazopanib, avapritinib, famitinib, catequentinib, necuparanib, surufatinib, lucitanib, midostaurin, vorolanib, bevasiranib, bevacizumab, ranibizumab, vanucizumab, navicixizumab, ramucirumab, BAT-5906, VGX-100, CSL-346, duvelisib, copanlisib, buparlisib, paxalisib, voxtalisib, zandelisib, dezapelisib, linperlisib, inavolisib, parsaclisib, eganelisib, nemiralisib, seletalisib, gedatolisib, leniolisib, tenalisib, pictilisib, bimiralisib, BBP-681, BGB-10188, MEN-1611, ASN-003, ACP-319, panobinostat, resminostat, abexinostat, romidepsin, belinostat, entinostat, quisinostat, pracinostat, tefinostat, mocetinostat, givinostat, dacinostat, ivaltinostat, domatinostat, fimepinostat, tinostamustine, remetinostat, tucidinostat, ricolinostat, CXD-101, REC-2282, veltuzumab, rituximab, ublituximab, nogitecan, simmitecan, gimatecan, topotecan, cositecan, belotecan, govitecan, deruxtecan, AR-67, camsirubicin, aldoxorubicin, vosaroxin, mitoxantrone, evofosfamide, amrubicin, sobuzoxane, epirubicin, F-14512, dacarbazine, temozolomide, nimustine, busulfan, procarbazine, melphalan, mitomycin C, daunorubicin, vincristine, vinblastine, vinorelbine, eribulin, trifluridine, docetaxel, cabazitaxel, avanbulin, fluorapacin, mertansine, carboplatin, nedaplatin, ixazomib, marizomib, carfilzomib and LXE-408.

### EXAMPLES

The following describes the present invention in more detail with reference to Examples; however, the present invention is not limited to these Examples. Although the present invention is sufficiently described in the Examples, a person skilled in the art would still be able to add various changes and/or modification thereto. Unless such changes and/or modification go beyond the scope of the present invention, the present invention encompasses such changes and/or modification.

The reagents used in the Examples are commercially available products, otherwise particularly indicated.

(i) X-Ray Powder Diffraction Measurement (XRPD Measurement) X-ray powder diffraction of a test substance was measured under the following test conditions after lightly pulverizing some amount of the test substance in an agate mortar as necessary.

Device: PANalytical: EMPYREAN

Target: Cu

X-ray output: 40 mA, 45 kV

Scanning Range: 5.0 to 40.0°

Step Size: 0.0131°

Scanning Speed: 0.0015°/sec.

Divergence Slit: 1°

Scattering Slit: 2.00 mm

Receiving Slit: 8.00 mm

The device and data were handled in accordance with the methods and procedures as instructed for the device.

(ii) Differential Scanning calorimetry Measurement (DSC Measurement)

DSC measurement was performed under the following conditions.

Device: METTLER TOLEDO: DSC1 STAR System

Sample: about 1 mg

Sample Container: made of aluminum

Temperature Range: 25 to 400° C.

Temperature Increase Rate: 10° C./min

Atmospheric Gas: Nitrogen

Nitrogen Gas Flow: 30 ml/min

The device and data were handled in accordance with the methods and procedures as instructed for the device.

(iii) High-Performance Liquid Chromatography

Measurement by high-performance liquid chromatography was performed under the following conditions.

Device: Shimadzu AC-20AD

UV detection: 220 nm

Flow Rate: 1 mL/min

Mobile Phase A: 10 mM phos. Buffer (pH6.3)/acetonitrile (95:5)

Mobile Phase B: acetonitrile

Column: Gemini C18 (4.6x 150 mm, 3 μm)/Phenomenex

Injection volume: 1 μL

Gradient:

| Retention time | Mobile Phase A | Mobile Phase B |
|---|---|---|
| (min) | (%) | (%) |
| 0 | 100 | 0 |
| 33 | 32 | 68 |
| 43 | 32 | 68 |
| 45 | 100 | 0 |
| 55 | 100 | 0 |

The device and data were handled in accordance with the methods and procedures as instructed for the device.

(iv) Liquid Chromatography/Mass spectrum (LCMS)

Measurement by LCMS was performed under the following conditions.

Device: Waters, SQD

MS detection: ESI positive

UV detection: 254 or 210 nm

Flow Rate: 0.5 mL/min Mobile Phase A: water

Mobile Phase B: acetonitrile, containing 0.1% formic acid

Column: Acquity BEH (2.1x 50 mm, 1.7 μm)

Injection volume: 1 μL

Gradient 1:

| Retention time | Mobile Phase A | Mobile Phase B |
|---|---|---|
| (min) | (%) | (%) |
| 0 | 95 | 5 |
| 0.1 | 95 | 5 |
| 2.1 | 5 | 95 |
| 3.1 | 5 | 95 |

Gradient 2:

| Retention time | Mobile Phase A | Mobile Phase B |
|---|---|---|
| (min) | (%) | (%) |
| 0 | 95 | 5 |
| 1.0 | 95 | 5 |
| 5.0 | 5 | 95 |
| 7.0 | 5 | 95 |

The device and data were handled in accordance with the methods and procedures as instructed for the device.

(v) Dynamic Vapor Sorption (DVS) System Analysis

Dynamic vapor sorption (DVS) system analysis was performed under the following conditions.

Apparatus: VTI SA+(manufactured by TA Instruments Inc.)

Drying temperature: 60° C.

Heating rate: 1° C./min

Drying equilibrium: Within the range not exceeding 300 minutes, the sample was confirmed not to decrease by 0.01 wt % in 5 minutes

Measurement temperature: 25° C.

Humidification equilibrium: Within the range not exceeding 120 minutes, the sample was confirmed not to increase by 0.01 wt % in 5 minutes

Relative Humidity Program: An increase by 5% RH from 5% RH to 95% RH and a decrease by 5% RH from 95% RH to 5% RH

A dedicated quartz holder was filled with 10 to 15 mg of crystal I of compound 1, and the weight of each sample at each humidity was continuously measured and recorded under the conditions described above. The device and data processing were handled in accordance with the method and procedure instructed for each device.

### Reference Example 1

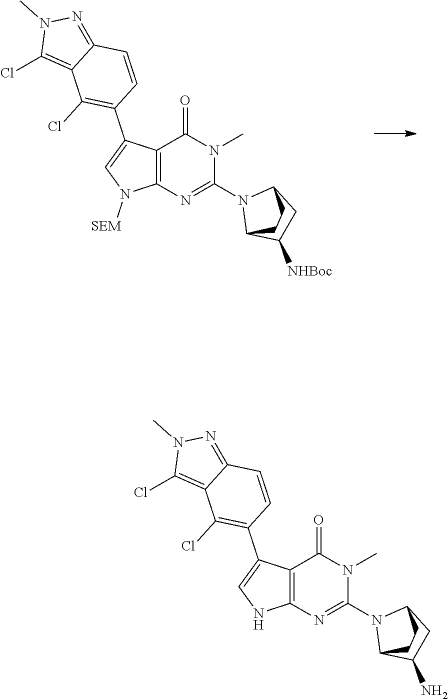 [SMILES: Cn1nc2ccc(-c3cn(COCC[Si](C)(C)C)c4nc(N5[C@H]6CC[C@@H]5[C@H](NC(=O)OC(C)(C)C)C6)n(C)c(=O)c34)c(Cl)c2c1Cl ; Cn1nc2ccc(-c3c[nH]c4nc(N5[C@H]6CC[C@@H]5[C@H](N)C6)n(C)c(=O)c34)c(Cl)c2c1Cl]

tert-Butyl ((1R,2R,4S)-7-(5-(3,4-dichloro-2-methyl-2H-indazol-5-yl)-3-methyl-4-oxo-74(2-(trimethylsilyl)ethoxy)methyl)-4,7-dihydro-3H-pyrrolo[2,3 d]pyrimidin-2-yl)-7-azabicyclo[2.2.1]heptan-2-yl)carbamate (44.7 g, 64.9 mmol) obtained as described in Patent Literature 1 was added to trifluoroacetic acid (274 g) at 0° C. The mixture was stirred at room temperature for 2 hours, and then added to a solution of ethylenediamine (195 g, 3.25 mol) in methanol (446 mL) at 0° C. The mixture was stirred at 50° C. for 4 hours. To the mixture, water (446 mL) was added at 0° C., then the mixture was stirred at room temperature for 6 hours. The precipitate was collected, washed with water, and dried at 50° C. under reduced pressure to give compound 1 (28.8 g).

### Example 1: Production of Crystal I of Compound 1

1-Propanol (20 mL) was added to compound 1 (1000 mg) obtained as described in Reference Example 1, and the mixture was stirred at 50° C. for 52.5 hours, followed by filtering the precipitate, thereby obtaining crystal I of compound 1 (890.5 mg).

As shown in Table 1 and [FIG. 1], crystal I exhibited an X-ray powder diffraction spectrum containing characteristic peaks at diffraction angles)(20±0.2° of 5.8°, 9.8°, 12.2°, 14.0°, 15.3°, 21.1°, and 27.6°.

TABLE 1

| | Diffraction | | | Relative |
|---|---|---|---|---|
| | Angles 2θ | d Value | Intensity | Intensity |
| No. | (°) | (Å) | (cps) | (%) |
| 1 | 5.7816 | 15.28649 | 445.65 | 65.6 |
| 2 | 8.5937 | 10.28955 | 20.44 | 3.0 |
| 3 | 9.8097 | 9.01671 | 68.05 | 10.0 |
| 4 | 12.1512 | 7.28396 | 156.06 | 23.0 |
| 5 | 12.8783 | 6.8743 | 7.69 | 1.1 |
| 6 | 13.2931 | 6.66069 | 109.77 | 16.2 |
| 7 | 14.0299 | 6.31252 | 401.55 | 59.2 |
| 8 | 15.2826 | 5.79779 | 251.77 | 37.1 |
| 9 | 16.2613 | 5.45098 | 144.59 | 21.3 |
| 10 | 17.216 | 5.15079 | 91.96 | 13.5 |
| 11 | 18.4067 | 4.8202 | 17.03 | 2.5 |
| 12 | 19.1337 | 4.63867 | 212.95 | 31.4 |
| 13 | 19.7083 | 4.50469 | 423.86 | 62.4 |
| 14 | 20.0033 | 4.43893 | 431.57 | 63.6 |
| 15 | 21.1049 | 4.20965 | 678.84 | 100.0 |
| 16 | 22.4007 | 3.96898 | 132.49 | 19.5 |
| 17 | 22.9674 | 3.87233 | 219.76 | 32.4 |
| 18 | 23.5557 | 3.77693 | 56.86 | 8.4 |
| 19 | 23.9939 | 3.70893 | 243.7 | 35.9 |
| 20 | 24.4534 | 3.64026 | 121.23 | 17.9 |
| 21 | 24.7322 | 3.59986 | 97.45 | 14.4 |
| 22 | 25.3567 | 3.5126 | 31.59 | 4.7 |
| 23 | 25.8468 | 3.44709 | 84.55 | 12.5 |
| 24 | 27.2922 | 3.26773 | 204.16 | 30.1 |
| 25 | 27.6206 | 3.22963 | 490.01 | 72.2 |
| 26 | 28.3083 | 3.15271 | 83.46 | 12.3 |
| 27 | 28.8009 | 3.0999 | 247.77 | 36.5 |
| 28 | 29.8289 | 2.99537 | 137.81 | 20.3 |
| 29 | 30.7532 | 2.90742 | 39.13 | 5.8 |
| 30 | 31.8093 | 2.81325 | 74.35 | 11.0 |
| 31 | 32.225 | 2.77791 | 106.14 | 15.6 |
| 32 | 33.4494 | 2.67897 | 16.94 | 2.5 |
| 33 | 34.2975 | 2.61464 | 64.68 | 9.5 |
| 34 | 34.6245 | 2.59069 | 64.69 | 9.5 |
| 35 | 35.2456 | 2.54645 | 39.32 | 5.8 |
| 36 | 35.9498 | 2.49817 | 3.97 | 0.6 |
| 37 | 36.6593 | 2.45144 | 55.32 | 8.1 |
| 38 | 37.3692 | 2.40648 | 47.75 | 7.0 |
| 39 | 37.7752 | 2.38155 | 51.2 | 7.5 |

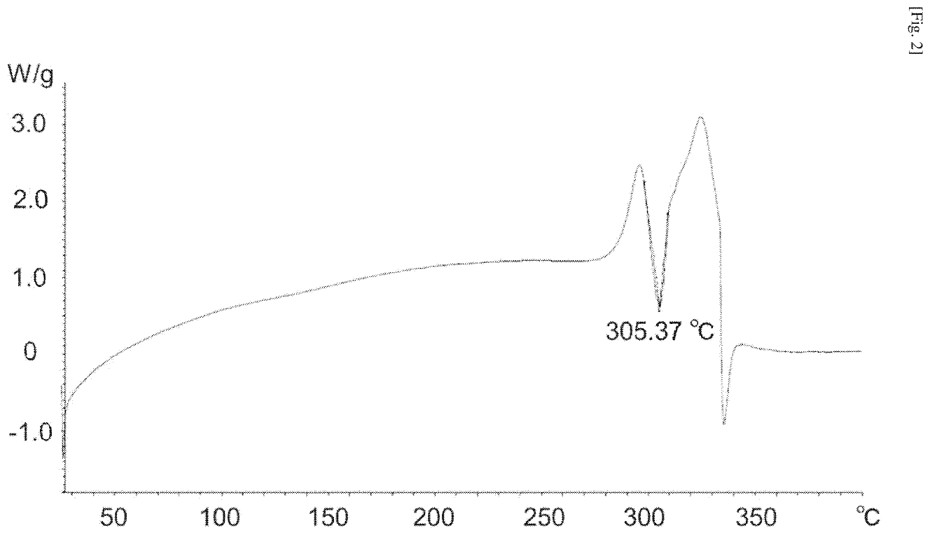

As shown in [FIG. 2], crystal I exhibited an endothermic peak (the highest peak value) in the vicinity of 305° C. in differential scanning calorimetry measurement (DSC measurement).

The chemical purity of crystal I was measured by LCMS.

The chemical purity of crystal I prepared in Example 1 was 99.54%.

### Example 2: Production of Crystal I of Compound 1

Compound 1 (54.0 g) obtained as described in Reference Example 1 was dissolved in DMSO (1300 mL) at ambient temperature. To the solution, water (1300 mL) was added at ambient temperature over 70 minutes, and the resulting suspension was stirred at ambient temperature for 100 minutes. Solid was collected by filtration, rinsed with 50% DMSO-water (250 mL) and then water (250 ml×2). The resulting solid was dried in vacuo at 50° C. over night to give crystal I of compound 1 (52.19 g).

Regarding the X-ray powder diffraction spectrum, the data of crystal I prepared in Example 2 basically exhibited the same as the data of crystal I prepared in Example 1.

The chemical purity of crystal I was measured by LCMS. The chemical purity of crystal I prepared in Example 2 was 99.56%.

### Test Example 1: Solid Stability of Crystal I of Compound 1

Crystal I of compound 1 was stored at 40° C. (humidity is not controlled), 40° C. (humidity 75%), and 60° C. (humidity is not controlled) for 4 weeks. Thereafter, the chemical purity was measured by high-performance liquid chromatography, and the change was 0.5% or less under all conditions. In differential scanning calorimetry measurement (DSC measurement) of Example 1, there was no peak indicating a phase transition even when the temperature was increased. The results indicate that crystal I of compound 1 has excellent solid stability.

### Test Example 2: Oral Absorbability of Crystal I of Compound 1

Crystal I of compound 1 was suspended in a 0.5% HPMC aqueous solution, and orally administered to male Sprague Dawley rats which were supplied from Charles River Laboratories Japan, Inc. in a dosage of 30 mg/kg. After 1, 2, 4, 6, 8, and 24 hours from administration, blood of each rat was collected from the left jugular vein, and the concentration of compound 1 in plasma was measured. The oral absorbability of crystal I of compound 1 was confirmed to have achieved a sufficient concentration that provides a medicinal effect.

TABLE 2

| | Crystal I |
|---|---|
| AUC0-24 μM · hr | 37 |
| Cmax μM | 3.5 |

### Test Example 3: Dynamic Vapor Sorption (DVS) System Analysis

Dynamic vapor sorption (DVS) system analysis was performed under the following conditions.

Apparatus: VTI SA+(manufactured by TA Instruments Inc.)

Drying temperature: 60° C.

Heating rate: 1° C./min

Drying equilibrium: Within the range not exceeding 300 minutes, the sample was confirmed not to decrease by 0.01 wt % in 5 minutes

Measurement temperature: 25° C.

Humidification equilibrium: Within the range not exceeding 120 minutes, the sample was confirmed not to increase by 0.01 wt % in 5 minutes

Relative Humidity Program: An increase by 5% RH from 5% RH to 95% RH and a decrease by 5% RH from 95% RH to 5% RH

A dedicated quartz holder was filled with 10 to 15 mg of crystal I of compound 1, and the weight of each sample at each humidity was continuously measured and recorded under the conditions described above. The device and data processing were handled in accordance with the method and procedure instructed for each device.

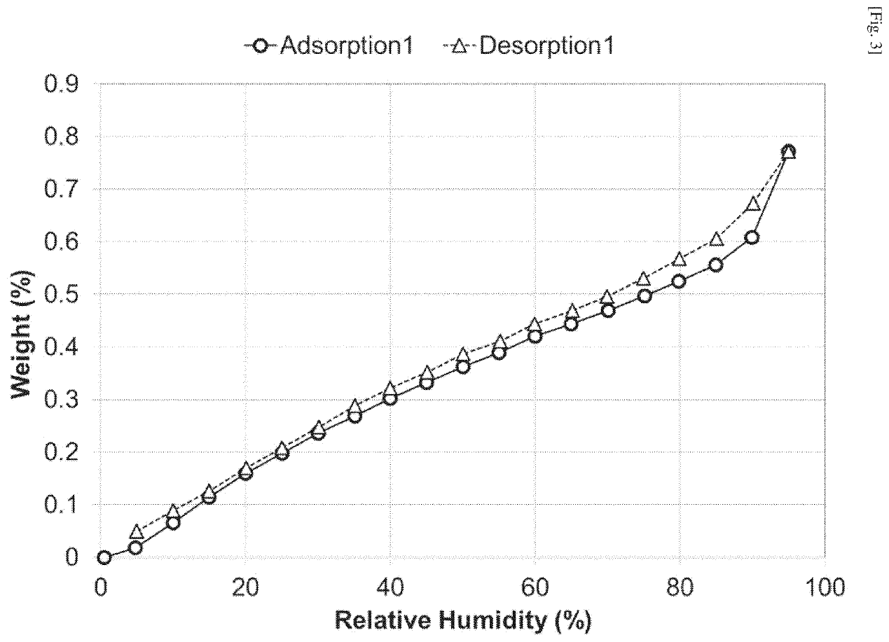

FIG. 3 shows the results of dynamic vapor sorption (DVS) system analysis with regard to crystal I.

There were no changes in weight due to changes in humidity in crystal I. Further, the results of powder X-ray crystal analysis on crystal I did not change before and after moisture adsorption and desorption.

1. A crystal of 2-41R,2R,4S)-2-amino-7-azabicyclo[2.2.1]heptan-7-yl)-5-(3,4-dichlor o-2-methyl-2Hindazol-5-yl)-3-methyl-3,7-dihydro-4H-pyrrolo[2,3-d]py rimidin-4-one exhibiting an X-ray powder diffraction spectrum containing, at diffraction angles 20±0.2°, at least 3 characteristic peak selected from the group consisting of 5.8°, 9.8°, 12.2°, 14.0°, 15.3°, 21.1°, and 27.6°.

2. The crystal according to claim 1, exhibiting an X-ray powder diffraction spectrum containing, at diffraction angles 20±0.2°, characteristic peaks of 5.8°, 9.8°, 12.2°, 14.0°, 15.3°, 21.1°, and 27.6°.

3. The crystal according to claim 1 or 2, which has a chemical purity of 98.0% or more.

4. The crystal according to any one of claims 1 to 3, which has an x-ray powder diffraction spectrum substantially as shown in FIG. 1.

5. The crystal according to any one of claims 1 to 3, which has an endothermic peak of 305±10° C. as measured by differential scanning calorimetry (DSC).

6. The crystal according to any one of claims 1 to 5, which has a DSC curve substantially as shown in FIG. 2.

7. The crystal according to any one of claims 1 to 6, which has a dynamic vapor sorption (DVS) curve substantially as shown in FIG. 3.

8. A pharmaceutical combination comprising a crystal according to any one of claims 1 to 7 and one or more other therapeutic agents.

9. A pharmaceutical combination according to claim 8, wherein the one or more other therapeutic agents are selected from anti-cancer agents or adjuvants (supporting agents in the therapy) in cancer therapy.

10. A pharmaceutical composition comprising the crystal according to any one of claims 1 to 7 or a pharmaceutical combination according to claim 8 or 9 and a pharmaceutically acceptable excipient.

11. The crystal according to any one of claims 1 to 7, pharmaceutical combination according to claim 8 or 9, or pharmaceutical composition according to claim 10 for use in medicine.

12. The crystal, pharmaceutical combination or pharmaceutical composition for use according to claim 11, for use in the treatment of cancer.

13. The crystal, pharmaceutical combination or pharmaceutical composition for use according to claim 12, wherein the cancer is head and neck cancer, esophagus cancer, gastric cancer, colon cancer, rectum cancer, liver cancer, gallbladder cancer, cholangiocarcinoma, biliary tract cancer, pancreatic cancer, lung cancer, breast cancer, ovarian cancer, cervical cancer, endometrial cancer, renal cancer, bladder cancer, prostate cancer, testicular tumor, osteosarcoma, soft-tissue sarcoma, leukemia, myelodysplastic syndrome, chronic myeloproliferative disease, malignant lymphoma, multiple myeloma, skin cancer, brain tumor, or mesothelioma.

14. A method for producing the crystal of any one of claims 1 to 7, comprising a crystallization step using at least one solvent selected from the group consisting of acetonitrile, methanol, ethanol, 1-propanol, 2-propanol, ethyl acetate, toluene, tetrahydrofuran, tert-butyl methyl ether, isopropyl ether, DMSO and water.

15. The method according to claim 14, wherein the crystallization step is performed using dimethyl sulfoxide as a good solvent and water as a poor solvent.
